(19) United States
(12) Patent Application Publication
(10) Pub. No.: US 2006/0057094 A1
(43) Pub. Date: Mar. 16, 2006

(54) Hair styling composition comprising at least one hydrophilic polymeric high porosity material and at least one fixing polymer

(76) Inventors: Estelle Mathonneau, Paris (FR); Isabelle Rollat, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/221,939

(22) Filed: Sep. 9, 2005

Related U.S. Application Data
(60) Provisional application No. 60/620,342, filed on Oct. 21, 2004.

(30) Foreign Application Priority Data
Sep. 9, 2004 (FR) 04 52007

(51) Int. Cl. A61K 8/81

(52) U.S. Cl. 424/70.16

(57) ABSTRACT

Described herein is a hair styling composition comprising, in a physiologically acceptable liquid medium: at least one hydrophilic polymeric high porosity material, and at least one fixing polymer, with the proviso that the at least one hydrophilic polymeric high porosity material is not a silicone polymer or an alkyl polymethacrylate polymer (PMMA). The compositions disclosed herein make it possible to obtain a more strongly fixed and a longer lasting hair style.

This application claims benefit of U.S. Provisional Application No. 60/620,342, filed Oct. 21, 2004, the contents of which are incorporated herein by reference. This application also claims benefit of priority under 35 U.S.C. § 119 to French Patent Application No. 04 52007, filed Sep. 9, 2004, the contents of which are also incorporated by reference.

Disclosed herein is a hair styling composition comprising at least one high porosity material and at least one fixing polymer.

Hair products come in a wide range of types, such as hair lacquers, sprays, gels or lotions. These products are polymer solutions or dispersions which, upon solvent phase evaporation, fix and hold the hair due to the presence of polymeric films that can envelop each hair with a sheath and the creation of physical links within the hair.

Hair styling products enable the user to model and fix the hair by means of fixing, tackifying, sticking, fat-enriched polymers and equivalents. These products should make it possible to fix the hair in a satisfactory and long-lasting way.

One of the problems associated with these hair styling products is their efficiency as time goes on.

To improve long-term efficiency, it is further known to permanently treat the keratinic fibers of the hair. These treatments may employ a reducing agent and an oxidizing agent and may need a mechanical hair tensioning by means of a rolling material so as to give the hair a durable shape.

Although these treatments do enable the user to fix the hair more durably than do gels associated with fixing polymers, such treatments suffer from the drawback of damaging the fiber feel and are relatively long-lasting methods.

The present inventors discovered that using high porosity polymeric materials associated with film-forming polymers makes it possible to obtain a more strongly fixed and longer lasting hair style.

Therefore, disclosed herein is a hair styling composition comprising, in a physiologically acceptable liquid medium:
at least one hydrophilic polymeric high porosity material, and
at least one fixing polymer,
with the proviso that the at least one hydrophilic polymeric high porosity material is not a silicone polymer or an alkyl polymethacrylate polymer (PMMA).

As used herein, the term “alkyl polymethacrylate type polymer (PMMA)” refers to homopolymers derived from polymerization reactions of methacrylate functionalities.

Hydrophilic Polymeric High Porosity Materials

As used herein the term “hydrophilic polymeric material” refers to a material comprising a polymer, at least one monomer of which is water-soluble.

As used herein, the term “water-soluble” monomer in the context of the present invention refers to a monomer which, upon introduction into water at a temperature of 25° C. and in a weight concentration of 0.5%, optionally neutralized, may produce a macroscopically homogenous and clear solution. In other words, the solution has a light transmittance value, at 500 nm wavelength, through a 1 cm thick sample, of at least 70%, for example, of at least 80%.

In at least one embodiment of the present disclosure, the high porosity polymeric material is solid, in the form of particles. The particles may have any form, for example, they may be spherical, elliptical, or polyhexagonal. The size of the hydrophilic polymeric material particles may range from 0.5 nm to 250 μm, for example, from 10 nm to 80 μm.

As used herein, the term “high porosity material” refers to a material which has a specific surface area greater than 0.1 m²/g. In one embodiment, the hydrophilic polymeric material may have a porosity such that specific surface area ranges from 0.1 to 1000 m²/g.

High porosity particles may absorb the polymer with which they are associated and create a coating onto the hair having “polymer sponges,” i.e., absorbent particles that may be impregnated with polymer and may significantly increase the amount of deposited polymer and the deposition quality, and hence, for a hair styling application, may improve the hair fixing. Such high porosity polymers may also give the cosmetic composition a special texture.

The hydrophilic monomers used in the present invention may be anionic, non-ionic, or cationic in nature and may be used alone or in mixtures of two or more different monomers.

Examples of anionic hydrophilic monomers include, but are not limited to, ethylenically unsaturated carboxylic acids such as acrylic acid, methacrylic acid, itaconic acid, fumaric acid, crotonic acid, maleic acid, 2-acrylamido-2-methylpropane-sulfonic acid, styrenesulfonic acid, vinylsulfonic acid, vinylphosphonic acid, and salts thereof.

Non-limiting examples of non-ionic hydrophilic monomers include acrylamide, C₁₋₆ N-alkylated acrylamides, C₁₋₃ N,N-dialkylated acrylamides, polyethylene glycol acrylate, polyethylene glycol methacrylate, N-vinyl acetamide, N-methyl-N-vinyl acetamide, N-vinyl formamide, N-methyl-N-vinyl formamide, N-vinyl lactames comprising a cyclic group comprising from 4 to 9 carbon atoms, vinyl alcohol (copolymerized as vinyl acetate, then hydrolyzed), ethylene oxide, hydroxyethyl acrylate, hydroxypropyl acrylate, hydroxyethyl methacrylate, hydroxypropyl methacrylate, methyl methacrylate, and N-acryloyl morpholine.

Examples of cationic hydrophilic monomers include, but are not limited to, dimethyldiallyl ammonium chloride, methylvinyl imidazolium chloride, 2-vinylpyridine, 4-vinylpyridine, 2-methyl-5-vinylpyridine, N—(C₁₋₄-alkyl)-4-vinylpyridinium halides such as N-methyl-4-vinylpyridinium iodide, vinyl amine, and monomers of formula:
H₂C═CR₁—CO—X₂
wherein:

R₁ is chosen from hydrogen and methyl,

X₂ is chosen from linear or branched C₁₋₆hydrocarbyl groups bearing at least one entity chosen from primary amine functionalities, secondary amine functionalities, tertiary amine functionalities, quaternary nitrogen atoms, groups of formula NHR₂, and groups of formula NR₂R₃, wherein R₂ and R₃ are independently chosen from linear or branched C₁₋₆ hydrocarbyl groups bearing at least one entity chosen from primary amine functionalities, secondary amine functionalities, tertiary amine functionalities, and quaternary nitrogen atoms.

The polymeric material may comprise from 0.1 to 40% of at least one hydrophobic monomer (water solubility at 25° C. of less than 0.5). The hydrophobic monomer may be chosen from vinyl aromatic monomers, such as styrene and alkylated derivatives thereof, for example, 4-butyl styrene, α-methyl styrene, and vinyl toluene; dienes, such as butadiene, 1,3-hexadiene, and diene alkylated derivatives, for example, isoprene and dimethyl butadiene; chloroprene; C₂-₁₀ alkyl acrylates; C₆₋₁₀ aryl acrylates; C₇₋₂₀ aralkyl acrylates; C₂₋₁₀ alkyl methacrylates; C₆₋₁₀ aryl methacrylates; C₇₋₂₀ aralkyl methacrylates, for example, methyl(meth)acrylates, ethyl(meth)acrylates, n-butyl(meth)acrylates, 2-ethylhexyl(meth)acrylates, tert-butyl(meth)acrylates, isobornyl(meth)acrylates, phenyl(meth)acrylates, benzyl(meth)acrylates, vinyl acetate, vinyl ethers of formula CH₂═CH—O—R, and allyl ethers of formula CH₂═CH—CH₂—O—R, wherein R is chosen from C₁₋₆ alkyl groups, acrylonitrile, vinyl chloride, vinylidene chloride, caprolactone, ethylene, propylene, fluorinated vinyl monomers, perfluorinated chain-vinyl monomers such as fluoralkyl acrylates and fluoroalkyl methacrylates, and α-fluoralkyl acrylates.

The hydrophilic polymeric high porosity material may be a homopolymer or a copolymer, including block copolymers.

Methods for preparing high porosity polymeric materials are described, for example, in U.S. Pat. Nos. 6,048,908 and 6,218,440. Such products are also described in International Application Publication No. WO 2003/091321 and in Caihua Ni-Zheng WANG & Xiao Xia ZHU, “Preparation and characterisation of thermosensitive beads with macroporous structures,” Journal of Applied Polymer Science, vol. 91, pp.1792-1797 (2004).

Fixing Polymers

As used herein, the term “fixing polymer” refers to any polymer that may shape the hair and/or retain a hair shape.

Any film-forming fixing polymer known in the hair care field may be used in the present invention, as well as mixtures thereof comprising more than one fixing polymer. The fixing polymers may be cationic, anionic, amphoteric, or non-ionic.

The cationic fixing polymers may be chosen from polymers comprising one or more primary, secondary, tertiary, and/or quaternary amine moieties belonging to the polymer chain or directly bound to it, and having molecular weights ranging from 500 to 5,000,000, for example, from 1,000 to 3,000,000.

Non-limiting examples of cationic polymers include:

(1) homopolymers and copolymers derived from at least one entity chosen from acrylic esters, methacrylic esters, acrylic amides, and methacrylic amides and comprising at least one of the following units: 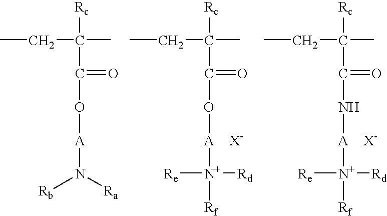
wherein:

Rₐ and R_{b}, which may be identical or different, are chosen from hydrogen and C₁₋₆ alkyl groups,

R_{c} is chosen from hydrogen and CH₃ radicals,

R_{d}, Rₑ, and R_{f}, which may be identical or different, are chosen from C₁₋₈ alkyl groups and benzyl radicals,

A is chosen from linear or branched C₁₋₆ alkyl groups and linear or branched C₁₋₄ hydroxyalkyl groups, and

X⁻ is an anion chosen from methosulfate and halide anions, for example, chloride and bromide ions.

Family (1) copolymers may further comprise at least one unit derived from comonomers chosen from acrylamides, methacrylamides, diacetone-acrylamides, acrylamides and methacrylamides substituted on the nitrogen by lower alkyl groups, acrylic acids, acrylic esters, methacrylic acids, methacrylic esters, vinyl lactames such as vinyl pyrrolidone and vinyl caprolactame, and vinyl esters.

Family (1) copolymers may include, for example:
acrylamide and dimethylaminoethyl methacrylate copolymers quaternized with dimethyl sulfate or with a dimethyl halide, such as the product marketed under the trade name HERCOFLOC® by HERCULES,
acrylamide and methacryloyl-oxyethyl trimethylammonium chloride copolymers, such as those described, for example, in European Pat. Application No. 0 080 976 and marketed under the trade name BINA QUAT® P 100 by CIBA GEIGY,
acrylamide and methacryloyl-oxyethyl trimethylammonium methosulfate copolymers marketed under the trade name RETEN® by HERCULES,
vinyl pyrrolidone/dialkylaminoalkyl acrylate or methacrylate copolymers optionally quaternized, such as products marketed under the trade name GAFQUAT® by ISP, for example, GAF-QUAT® 734 and GAFQUAT® 755, or products called COPOLYMER®845, 958, and 937. These polymers are described, for example, in French Patent Applications 2 077 143 and 2 393 573,
dimethylaminoethyl methacrylate/vinyl caprolactame/vinyl pyrrolidone terpolymers such as the product marketed under the trade name GAFFIX VC 713 by ISP, and
vinyl pyrrolidone/dimethylaminopropyl methacrylamide quaternized copolymers such as the product marketed under the trade name GAFQUAT® HS 100 by ISP.

(2) quaternized polysaccharides, such as those described in U.S. Pat. Nos. 3,589,578 and 4,031,307,for example, guar gums comprising cationic trialkyl ammonium moieties. Examples of commercial products include those marketed under the trade names JAGUAR® C13 S, JAGUAR® C15, and JAGUAR® C17 by MEYHALL.

(3) vinyl pyrrolidone and vinyl imidazole quaternary copolymers such as the products marketed by BASF under the trade name LUVIQUAT® TFC,

(4) chitosans and salts thereof, for example, chitosan acetate, lactate, glutamate, gluconate, and pyrrolidone carboxylate. Non-limiting example of chitosans which may be used in accordance with the present disclosure include chitosan having a 90.5% by weight deacetylation rate, marketed under the trade name KYTAN BRUT STANDARD® by ABER TECHNOLOGIES, and chitosan pyrrolidone carboxylate marketed under the trade name KYTAMER® PC by AMERCHOL.

(5) cellulose cationic derivatives such as cellulose copolymers or cellulose derivatives grafted onto a water-soluble monomer comprising a quaternary ammonium group, such as those described, for example, in U.S. Pat. No. 4,131,576, for example, hydroxyalkyl celluloses, hydroxymethylhydroxyethyl or hydroxypropyl celluloses grafted onto an entity chosen from methacryloyloxyethyltrimethyl ammonium salts, methacrylamidopropyl-trimethyl ammonium salts, and dimethyldiallyl ammonium salts. Examples of commercial products that correspond to these derivativdes include products marketed under the trade names CELQUAT® L 200 and CELQUAT®H 100 by NATIONAL STARCH.

Anionic fixing polymers may be chosen from polymers comprising moieties derived from a carboxylic, sulfonic, and/or phosphoric acid, and having a weight average molecular weight ranging from 500 to 5,000,000.

Carboxylic acid moieties may be provided by unsaturated monomers comprising one or two carboxylic acid functionalities, such as those of formula I: 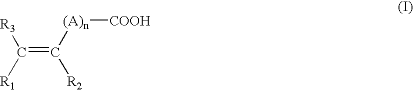
wherein:

n is an integer ranging from 0 to 10,

A is a methylene moiety, optionally linked to the carbon atom of the adjacent unsaturated moiety or methylene moiety, when n is more than 1, through a heteroatom such as oxygen or sulfur,

R₃ is chosen from phenyl and benzyl moieties,

R₁ is chosen from hydrogen, lower alkyl moeties, and carboxyl moieties, and

R₂ is chosen from hydrogen, lower alkyl moieties, —CH₂—COOH moieties, phenyl moieties, and benzyl moieties.

In the above formula, a lower alkyl radical may refer to a moiety comprising from 1 to 4 carbon atoms, for example, methyl and ethyl groups.

Examples of anionic fixing polymers containing carboxyl groups include:

A) acrylic acid homopolymers, acrylic acid copolymers, methacrylic acid homopolymers, methacrylic acid copolymers, and salts thereof, for example, the products marketed under the trade names VERSICOL® E and VERSICOL® K by ALLIED COLLOID, and under the trade name ULTRAHOLD® by BASF; acrylic acid and acrylamide copolymers sold as sodium salt under the trade names RETEN® 421, 423, and 425 by HERCULES; and sodium salts of polyhydroxycarboxylic acids.

B) copolymers of acrylic acid or methacrylic acid with a monoethylenic monomer chosen from, for example, ethylene, styrene, vinyl esters, acrylic acid esters, and methacrylic acid esters.

These copolymers may be grafted onto a polyalkylene glycol such as polyethylene glycol and may optionally be crosslinked.

Such polymers are described, for example, in French Patent Application No. 1 222 944 and German Patent Application No. 2 330 956. Copolymers that comprise an optionally N-alkylated and/or hydroxyalkylated acrylamide unit in their chain, such as those described in the Luxemburg Patent Application Nos. 75370 and 75371 and those proposed under the trade name QUADRAMER® by AMERICAN CYANAMID are also suitable for use in accordance with the present disclosure.

Other examples of copolymers include acrylic acid and C₁-C₄ alkyl methacrylate copolymers; vinyl pyrrolidone, (meth)acrylic acid, and C₁-C₂₀ alkyl (meth)acrylate terpolymers, for example those based on lauryl (ACRYLDONE® LM from ISP), those based on tert-butyl (LUVIFLEX® VBM 70 marketed by BASF), and those based on methyl (STEPANHOLD® EXTRA marketed by STEPAN); and methacrylic acid/ethyl acrylate/tert-butyl acrylate terpolymers such as the product marketed under the trade name LUVIMER® 100 P by BASF.

C) Copolymers derived from crotonic acid such as those comprising at least one unit chosen from vinyl acetate and propionate units in their chain, and optionally other monomers such as allyl, methallyl, and vinyl esters of a saturated, linear or branched, carboxylic acid, with a hydrocarbyl long chain comprising at least 5 carbon atoms, these polymers being optionally grafted and cross-linked, and vinyl, allyl, or methallyl esters of a α- or β-cyclic carboxylic acid. Such polymers are described, for example, in French Patent Application Nos. 1 222 944, 1 580 545, 2 265 782, 2 265 781, 1 564 110, and 2 439 798. Resins 28-29-30, 26-13-14, and 28-13-10 marketed by NATIONAL STARCH are examples of commercial products belonging to this class.

D) Copolymers derived from entities chosen from C₄-C₈ mono-unsaturated carboxylic acids and C₄-C₈ mono-unsaturated carboxylic anhydrides chosen from:
copolymers comprising:
(i) at least one entity chosen from maleic acid, maleic anhydride, fumaric acid, fumaric anhydride, itaconic acid, and itaconic anhydride, and
(ii) at least one monomer chosen from vinyl esters, vinyl ethers, vinyl halides, phenyl vinyl derivatives, acrylic acid, acrylic esters, the anhydride functionalities of these copolymers being optionally monoesterified or monoamidified.

Such polymers are described, for example, in U.S. Pat. Nos. 2,047,398, 2,723,248, and 2,102,113 and British Patent No. 839,805. Examples of commercial products include, but are not limited to, those marketed under the trade names GANTREZ AN, GANTREZ ES, and AVANTAGE® CP by ISP;
copolymers comprising:
(i) at least one entity chosen from maleic, citraconic, and itaconic anhydrides, and
(ii) at least one monomer chosen from allyl and methallyl esters optionally comprising at least one moiety chosen from acrylamide, methacrylamide, α-olefin, acrylic esters, methacrylic esters, acrylic acid, methacrylic acid, and vinyl pyrrolidone moieties in their chain, the anhydride functionalities of these copolymers being optionally monoesterified or monoamidified.

These polymers are described, for example, in French Patent Application Nos. 2 350 384 and 2 357 241.

E) Polyacrylamides comprising carboxylate moieties.

The anionic moieties of the anionic fixing polymers of the present disclosure may also be chosen from sulfonic acid groups provided by at least one unit chosen from vinylsulfonic, styrenesulfonic, naphtalenesulfonic, and acrylamidoalkylsulfonic units.

These polymers containing sulfonic acid groups may be chosen from:
poly(vinylsulfonic acid) salts having a weight average molecular weight ranging from 1,000 to 100,000, and copolymers of vinylsulfonic acid with an unsaturated comonomer such as acrylic acid, methacrylic acid, esters of these acids, acrylamide, acrylamide derivatives, vinyl ethers, and vinyl pyrrolidone;
poly(styrenesulfonic acid) salts, for example, two sodium salts having a weight average molecular weight ranging from 500,000 to 100,000, respectively marketed under the trade names FLEXAN® 500 and FLEXAN® 130 by NATIONAL STARCH. These compounds are described, for example, in French Patent No. 2 198 719; and
poly(acrylamidesulfonic acid)salts, such as those described in U.S. Pat. No. 4,128,631 and the poly(acrylamidoethyl-propanesulfonic acid) marketed under the trade name COSMEDIA POLYMER® HSP 1180 by HENKEL.

Further non-limiting examples of anionic fixing polymers include acrylic acid copolymers such as acrylic acid/ethyl acrylate/N-tert-butylacrylamide terpolymers such as those marketed under the trade name ULTRAHOLD STRONG® by BASF; crotonic acid derived copolymers such as vinyl acetate/vinyl tert-butyl benzoate/crotonic acid terpolymers and crotonic acid/vinyl acetate/vinyl neododecanoate terpolymers marketed under the trade name Résine 28-29-30 by NATIONAL STARCH; copolymers derived from at least one entity chosen from maleic acid, maleic anhydride, fumaric acid, fumaric anhydride, itaconic acid, and itaconic anhydride, comprising comonomers chosen from vinyl esters, vinyl ethers, vinyl halides, phenyl vinyl derivatives, acrylic acid, and acrylic acid esters, such as methylvinyl ether/monoesterified maleic anhydride copolymers, marketed, for example, under the trade name GANTREZ® by ISP, methyl methacrylate acid copolymers marketed under the trade name EUDRAGIT®L by ROHM PHARMA, methacrylic acid/methyl methacrylate/C₁₋₄ alkyl acrylate/acrylic acid or C₁₋₄ hydroxyalkyl methacrylate copolymers marketed under the trade name AMERHOLD® DR 25 by AMERCHOL, or under the trade name ACUDYNE® 255 by ROHM & HAAS, methacrylic acid and ethyl acrylate copolymers marketed under the trade names LUVIMER® MAEX and LUVIMER® MAE by BASF and vinyl acetate/crotonic acid copolymers and vinyl acetate/crotonic acid copolymers grafted with polyethylene glycol marketed under the trade name ARISTOFLEX® A by BASF.

Amphoteric fixing polymers which may be used in accordance with the present disclosure include, but are not limited to, polymers comprising B and C units statistically distributed in the polymer chain, where B is a unit derived from a monomer comprising at least one basic nitrogen atom and C is a unit derived from a monomer comprising at least one moiety chosen from carboxylic acid and sulfonic acid moieties. Amphoteric fixing polymers may also comprise carboxy betaine and/or sulfobetaine zwitterionic units. The amphoteric fixing polymers may comprise a cationic main chain comprising primary, secondary, tertiary, and/or quaternary amine moieties, amongst which at least one bears at least one moiety chosen from carboxylic acid and sulfonic acid moieties through a hydrocarbyl radical. Amphoteric fixing polymers may further have an anionic chain derived from α,β-unsaturated dicarboxylic acids, one carbonyl moiety of which has been reacted with a polyamine comprising at least one primary or secondary amine moiety.

Non-limiting examples of amphoteric fixing polymers include:
(1) Polymers resulting from the copolymerization of at least one vinyl monomer bearing a carboxylic acid moiety, such as acrylic acid, methacrylic acid, maleic acid, α-chloroacrylic acid, and of at least one vinyl monomer comprising at least one basic functionality, such as dialkylaminoalkyl methacrylate and -acrylate and dialkylaminoalkyl (meth)acrylamides. Such compounds are described, for example, in U.S. Pat. No. 3,836,537.
(2) Polymers comprising units derived from:
   (a) at least one monomer chosen from N-alkylated acrylamides and methacrylamides,
   (b) at least one comonomer comprising at least one carboxylic acid functionality, and
   (c) at least one basic comonomer, such as esters substituted with a primary, secondary, tertiary, and/or quatemary amine of acrylic acid and methacrylic acid, and the quatemization product of dimethylaminoethyl methacrylate and dimethyl- or diethylsulfate.

Preferred N-alkylated acrylamides and methacrylamides are those bearing C₂₋₁₂ alkyl radicals such as N-ethylacrylamide, N-tert-butylacrylamide, N-tert-octylacrylamide, N-octylacrylamide, N-decylacrylamide, N-dodecylacrylamide, N-ethylmethacrylamide, N-tert-butylmethacrylamide, N-tert-octylmethacrylamide, N-octylmethacrylamide, N-decylmethacrylamide, and N-dodecylmethacrylamide.

Comonomers comprising carboxylic acid groups may be choen from acrylic acid, methacrylic acid, crotonic acid, itaconic acid, maleic acid, fumaric acid, and C₁₋₄ alkyl monoesters of at least one entity chosen from maleic acid, maleic anhydride, fumaric acid, and fumaric anhydride.

Basic comonomers include, but are not limited to, aminoethyl methacrylate, butylaminoethyl methacrylate, N,N′-dimethylaminoethyl methacrylate, and N-tert-butylaminoethyl methacrylate.

In at least one embodiment, the amphoteric copolymer has the CTFA name (4^{th} Ed., 1991) “Octylacrylamide/acrylates/butylaminoethyl methacrylate copolymer”, for example, the products marketed under the trade names AMPHOMERO° and LOVOCRYL° 47 by NATIONAL STARCH.

(3) Cross-linked and alkylated polyaminoamides, fully or partially derived from polyaminoamides of formula (II):
—[C(=0)-R₄—C(=0)-Z-]- (II)
wherein R₄ is chosen from divalent radicals derived from an entity chosen from saturated dicarboxylic acid, monocarboxylic aliphatic acids with an ethylene double bond, dicarboxylic aliphatic acids with an ethylene double bond, C₁₋₆ alkyl esters of these acids, radicals resulting from the addition of any one of said acids onto a bis-primary or bis-secondary amine, and Z is chosen from bis-primary, mono-secondary, and bis-secondary polyalkylene-polyamine radicals, for example:

a) in amounts ranging from 60 to 100 mol %, a radical of formula (III):
—NH—[(CH₂)ₓ—NH]ₚ— (III)
wherein x=1 and p=2 or 3, or x=3 and p=2, this radical being derived from an entity chosen from diethylene triamine, triethylene tetraamine, and dipropylene triamine;

b) in amounts ranging from 0 to 40 mol %, a radical of formula (III), where x=2 and p=1, derived from ethylene diamine, or the radical 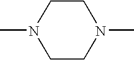
derived from piperazine;

c) in amounts ranging from 0 to 20 mol %, a radical —NH—(CH₂)₆—NH— derived from hexamethylene diamine, these polyaminoamines being cross-linked by adding from 0.025 to 0.35 mol % of the amine moiety of a difunctional cross-linking agent chosen from epihalohydrines; diepoxides; dianhydrides; di-unsaturated compounds alkylated with an alkylation agent chosen from acrylic acid, chloroacetic acid, and alkane-sulfones.

Saturated carboxylic acids may be chosen from, for example, acids comprising from 6 to 10 carbon atoms, such as adipic acid, trimethyl-2,2-4-adipic acid, trimethyl-2,4,4-adipic acid, terephtalic acid, and ethylene double-bonded acids, for example, acrylic, methacrylic, and itaconic acids.

Alkane-sulfones used for alkylation may be chosen from sulfone and butane sulfone. Alkylation agent salts may be chosen from sodium or potassium salts.

(4) Polymers comprising zwitterionic units of formula (IV): 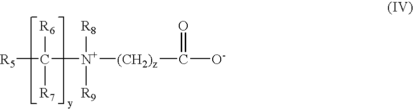
wherein:

R₅ is chosen from polymerizable unsaturated moieties such as acrylate, methacrylate, acrylamide, and methacrylamide moieties,

y and z, which may be identical or different, are integers ranging from 1 to 3,

R₆ and R₇, which may be identical or different, are chosen from hydrogen, methyl groups, ethyl groups, and propyl groups, and

R₈ and R₉, which may be identical or different, are chosen from hydrogen and alkyl radicals, with the proviso that the total number of carbon atoms in R₈ and R₉ is not greater than 10.

Polymers comprising units of formula (IV) may further comprise units derived from non-zwitterionic monomers, such as dimethyl ethyl acrylate, dimethyl ethyl methacrylate, diethylamino ethyl acrylate, and diethylamino ethyl methacrylate, alkyl acrylates, alkyl methacrylates, acrylamides, methacrylamides, and vinyl acetate.

A non-limiting example of a polymer comprising units of formula (IV) is methyl methacrylate/dimethyl carboxymethyl ammonioethyl methacrylate copolymer, such as the product marketed under the trade name DIAFORMER® Z301 by SANDOZ.

(5) Polymers derived from chitosan, comprising at least one monomer unit chosen from units of formulas (V), (VI), and (VII): 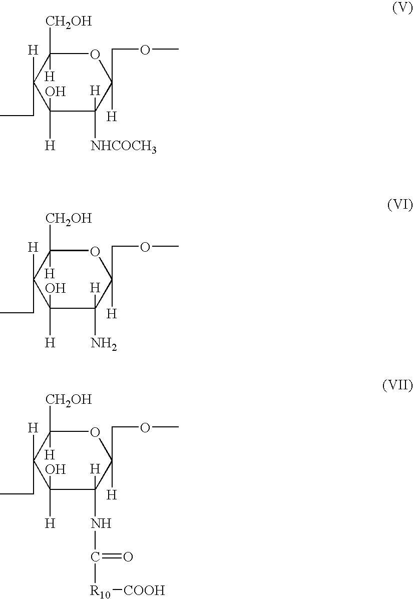
wherein the units of formula (V) are present in amounts ranging from 0 to 30%, units of formula (VI) are present in amounts ranging from 5 to 50%, and units of formula (VII) are present in amounts ranging from 30 to 90%, and wherein R₁₀ represents a radical of formula (VIII): 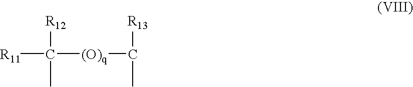
wherein:

when q=0, then R₁₁, R₁₂, and R₁₃, which may be identical or different, are chosen from hydrogen; methyl groups; hydroxyl groups; acetoxy groups; amino groups; monoalkyl amine or dialkyl amine groups optionally interrupted by at least one nitrogen atom and/or optionally substituted by at least one group chosen from amine, hydroxyl, carboxyl, alkylthio, and sulfo groups; and alkylthio groups in which the alkyl group bears an amino radical, and at least one of R₁₁, R₁₂, and R₁₃ is hydrogen; and

when q=1, then R₁₁, R₁₂, and R₁₃ are hydrogen,

as well as the acid and base addition salts formed by these compounds.

6) Polymers obtained by chitosan N-carboxyalkylation, such as N-carboxymethylchitosan and N-carboxybutylchitosan marketed under the trade name EVALSAN® by JAN DEKKER.

(7) Polymers of formula (IX): 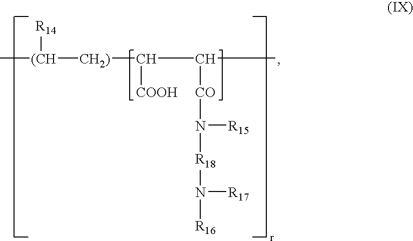
for example, those described in the French Patent Application No. 1 400 366, wherein

R₁₄ is chosen from hydrogen, CH₃O, CH₃CH₂O, and phenyl radicals,

R₁₅ and R₁₆, which may be identical or different, are chosen from hydrogen and lower alkyl radicals such as methyl and ethyl radicals,

R₁₇ is chosen from lower alkyl radicals such as methyl and ethyl radicals and radicals of the following formula:
—R₁₈—N(R₁₆)₂,
wherein:

R₁₈ is chosen from —CH₂—CH₂—,—CH₂—CH₂—CH₂—, and —CH2-CH(CH₃)— moieties, and

R₁₆ is defined above, as well as higher homologs of these radicals, comprising up to 6 carbon atoms.

(8) Amphoteric polymers of -D-X-D-X- type chosen from:
(a) polymers obtained by reacting chloroacetic acid or sodium chloroacetate with compounds comprising at least one unit of formula:
   (X)-D-X-D-X-D-
   wherein D is a radical: 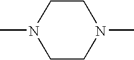
   and X is chosen from E and E′, which may be identical or different, wherein E and E′ are independently chosen from straight or branched chain alkylene divalent radicals comprising up to 7 carbon atoms in the main chain, unsubstituted or substituted by hydroxyl moieties, and optionally further comprising at least one entity chosen from oxygen, nitrogen, and sulfur, from 1 to 3 aromatic and/or heterocyclic rings, wherein the oxygen, nitrogen, and sulfur atoms may be present in the form of ether, thioether, sulfoxide, sulfone, sulfonium, alkyl amine, alcenyl amine moieties, hydroxyl, benzyl amine, amine oxide, quaternary ammonium, amide, imide, alcohol, ester, and/or urethane moieties.
b) Polymers of the following formula:
   (X′)-D-X′-D-X′-
   where D is a radical 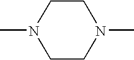
   and X′ is chosen from E or E′, and at least once E′, wherein E is defined as above and E′ is chosen from straight or branched chain divalent alkylene radicals comprising up to 7 carbon atoms in the main chain, optionally substituted by at least one hydroxyl radical and comprising at least one nitrogen atom, the nitrogen atom being substituted by an alkyl chain optionally interrupted by an oxygen atom and comprising at least one functionality chosen from carboxyl and hydroxyl functionalities, and being betainized by reaction with chloroacetic acid or sodium chloroacetate.

9) (C₁₋₅)alkyl vinyl ether/maleic anhydride copolymers partially modified by semi-amidification with an N,N-dialkylaminoalkylamine such as N,N-dimethylaminopropylamine, or by semi-esterification with an N,N-dialkanolamine. These copolymers may also comprise other vinyl comonomers such as vinyl caprolactame.

In at least on embodiment, the amphoteric fixing polymer is chosen from those of family (3), such as those having the CTFA name “Octylacrylamide/acrylates/butyl-aminoethyl-methacrylate copolymer.” Examples of commercial products include those marketed under the trade names AMPHOMER®, AMPHOMER® LV 71, and LOVOCRYL® 47 by NATIONAL STARCH.

In another embodiment, the amphoteric fixing polymer is chosen from those of family (4), for example, methyl methacrylate and dimethylcarboxymethyl ammonioethyl methacrylate copolymers, marketed, for example, under the trade name DIAFORMER® Z301 by SANDOZ.

Anionic or amphoteric fixing polymers may, in at least one embodiment, be partially or fully neutralized. Neutralizing agents may include, for example, soda, potash, amino-2-methylpropanol, monoethanolamine, triethanolamine, tri-isopropanolamine, and inorganic or organic acids such as hydrochloric acid and citric acid.

Non-ionic fixing polymers which may be used according to the present disclosure include, but are not limited to:
vinyl pyrrolidone homopolymers,
vinyl pyrrolidone and vinyl acetate copolymers,
polyalkyl oxazolines such as polyethyl oxazolines marketed by DOW CHEMICAL under the trade names PEOX® 50 000, PEOX® 200 000, and PEOX® 500 000,
vinyl acetate homopolymers such as the product marketed under the trade name APPRETAN® EM by HOECHST or the product marketed under the trade name RHODOPAS® A 012 by RHONE POULENC;
vinyl acetate and acrylic ester copolymers such as the product marketed under the trade name RHODOPAS® AD 310 by RHONE POULENC,
vinyl acetate and ethylene copolymers such as the product marketed under the trade name APPRETAN® TV by HOECHST,
vinyl acetate and maleic ester copolymers, for example, those based on dibutyl maleate, such as the product marketed under the trade name APPRETAN® MB EXTRA by HOECHST,
ethylene and maleic anhydride copolymers,
alkyl acrylate homopolymers and alkyl methacrylate homopolymers such as the product marketed under the trade name MICROPEARL® RQ 750 by MATSUMOTO or the product marketed under the trade name LUHYDRAN® A 848 S by BASF,
acrylic ester copolymers, for example, alkyl acrylate and alkyl methacrylate copolymers such as the products marketed by ROHM & HAAS under the trade names PRIMAL AC-261 K and EUDRAGIT NE 30 D, by BASF under the trade names ACRONAL® 601, LUHYDRAN® LR 8833, and LUHYDRAN® LR 8845, and by HOECHST under the trade names APPRETAN® N 9213 and N 9212,
copolymers of acrylonitrile and a non-ionic monomer chosen from, for example, butadiene and alkyl (meth)acrylates; such as the products marketed under the trade names NIPOL® LX 531 B by NIPPON ZEON or those proposed under the trade name CJ 0610 B by ROHM & HAAS,
polyurethanes such as the products marketed under the trade names ACRYSOL® RM 1020 and ACRYSOL® RM 2020 by ROHM & HAAS, the products URAFLEX® XP 401 UZ, URAFLEX® XP, and 402 UZ by DSM RESINS,
alkyl acrylate and urethane copolymers such as the product 8538-33 marketed by NATIONAL STARCH,
polyamides such as the product ESTAPOR® LO 11 marketed by RHONE POULENC, and
non-ionic guar gums which are optionally chemically modified.

Examples of non-ionic, non-modified guar gums include, for example, the products marketed under the trade name VIDOGUM® GH 175 by UNIPECTINE and under the trade name JAGUAR® C by MEYHALL. Non-ionic, modified guar gums which may be used according to the present disclosure may be modified by C₁₋₈ hydroxyalkyl moieties, for example, hydroxymethyl, hydroxyethyl, hydroxypropyl, and hydroxybutyl moieties.

These guar gums are well known in the art and may be prepared, for example, by reacting corresponding alkene oxides, such as propylene oxides, with the guar gum so as to obtain a guar gum modified by a hydroxypropyl moiety.

Such non-ionic guar gums, optionally modified with hydroxyalkyl moieties are marketed, for example, under the trade names JAGUAR® HP8, JAGUAR® HP60, JAGUAR® HP120, JAGUAR® DC 293, and JAGUAR® HP 105 by MEYHALL, or under the trade name GALACTASOL® 4H4FD2 by AQUALON.

According to the present disclosure, graft silicone-type film-forming polymers may also be used as fixing polymers, comprising a polysiloxane part and a non-silicone organic chain part, one of the two parts forming the polymer main chain and the other one being graft onto said main chain.

These polymers are described, for example, in European Patent Nos. 0 412 704, 0 412 707, 0 640 105, and 0 582 152, International Patent Publication Nos. WO 93/23009 and WO 95/00578, and U.S. Pat. Nos. 4,693,935, 4,728,571, and 4,972,037.

In at least one embodiment, these polymers may be anionic or non-ionic in nature.

Such polymers may include, for example, copolymers that may be prepared by free radical polymerization of a monomer mixture comprising,

a) from 50 to 90% by weight of tert-butyl acrylate,

b) from 0 to 40% by weight of acrylic acid,

c) from 5 to 40% by weight of a silicone macromer of following formula: 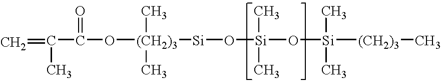
wherein v is a number ranging from 5 to 700, and wherein the weight percentages are based on the monomer total weight.

Other silicone graft-polymers include, for example, polydimethyl siloxanes (PDMS), onto which mixed poly(meth)acrylic and alkyl poly(meth)acrylate polymer units are grafted through a thiopropylene chain linkage, and polydimethyl siloxanes (PDMS) onto which isobutyl poly(meth)acrylate polymer units are grafted through a thiopropylene chain linkage.

Optionally functionalized, silicone or non-silicone polyurethanes may also be used as film-forming fixing polymers in accordance with the present disclosure.

Non-limiting examples of polyurethanes include those described in European Patent Application Nos. 0 751 162, 0 637 600, 2 743 297, 0 648 485, 0 656 021, and 0 619 111 and International Publication No. WO 94/03510.

The hair styling composition according to the invention may also comprise at least one compound selected from silicones in soluble, dispersed, micro- or nanodispersed, volatile or non-volatile form, thickening agents, non-ionic surfactants, anionic surfactants, cationic surfactants, amphoteric surfactants, conditioning agents, softening agents, anti-foam agents, moisturizing agents, emollients, plasticizers, water-soluble or oil-soluble, silicone or non-silicone sunscreens, permanent dyes, temporary dyes, organic or inorganic pigments which may or may not be colored, inorganic fillers, clays, pearlescent agents, opacifying agents, colloids, fragrances, peptizing agents, preserving agents, ceramides, pseudo-ceramides, vitamins, provitamins such as panthenol, proteins, sequestering agents, solubilizing agents, alkalizing agents, anti-corrosion agents, fats such as vegetable, animal, mineral, and synthetic oils, reducing agents, antioxidants, oxidants, and mixtures thereof.

In at least one embodiment, the physiologically acceptable liquid medium may be chosen from water, at least one organic solvent, and mixtures of water and at least one organic solvent.

The organic solvent may be selected from C₁-C₄ alcohols, C₅-C₁₀ alkanes, fatty alcohols, optionally modified polyvalent alcohols, volatile and non-volatile silicones, mineral oils, organic oils, vegetal oils, and mixtures thereof.

The cosmetic composition according to the invention may be a leave-on composition (no need for rinse-off) and may be provided in a form chosen from sprays, foams, and gels. When applying the composition to the hair, the hair may be heated before, during, or after the application of the composition.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below.

### EXAMPLE 1

A composition of formula 1 according to the disclosure and a comparative composition of formula 2 were prepared by combining the following components:

Formula 1

| | |
|---|---|
| Carbomer synthalen K | 0.7% |
| Acrylamide-based porous sphere | 4.0% |
| described in example 1 of patent WO 2003/091 321 | |
| Fixate g100 | 3.0% |
| Water | 92.3% |

Formula 2

| | |
|---|---|
| Carbomer synthalen K | 0.7% |
| Fixate g100 | 3.0% |
| Water | 96.3% |

1 g of formula (1) was applied to 2.5 g of European type brown hair after the hair was dried. 1 g of control formula (2) was applied to 2.5 g of European type brown hair of the same nature after the hair was dried.

It was observed, based on a visual qualitative comparison, that after the hair had been submitted to a horizontal oscillating movement for time periods of half an hour, two hours, and half a day, the hair strand fixed with formula (1), gave the hair strand the best shape and the longest lasting hold.

What is claimed is:

1. A hair styling composition comprising, in a physiologically acceptable liquid medium:
at least one hydrophilic polymeric high porosity material, and
at least one fixing polymer,
with the proviso that the at least one hydrophilic polymeric high porosity material is not a silicone polymer or an alkyl polymethacrylate polymer (PMMA).

2. A hair styling composition according to claim 1, wherein the at least one hydrophilic polymeric high porosity material is a solid, in the form of particles.

3. A hair styling composition according to claim 2, wherein the at least one hydrophilic polymeric material particle size ranges from 0.5 nm to 250 μm.

4. A hair styling composition according to claim 3, wherein the at least one hydrophilic polymeric material particle size ranges from 10 nm to 80 μm.

5. A hair styling composition according to claim 1, wherein the at least one hydrophilic polymeric material has a specific surface area ranging from 0.1 to 1,000 m²/g.

6. A hair styling composition according to claim 1, wherein the hydrophilic polymeric high porosity material comprises at least one hydrophilic monomer chosen from anionic monomers, non-ionic monomers, cationic monomers, and mixtures thereof.

7. A hair styling composition according to claim 6, wherein the hydrophilic anionic monomers are selected from ethylenically unsaturated carboxylic acids, 2-acrylamido-2-methylpropane-sulfonic acid, styrenesulfonic acid, vinylsulfonic acid, vinylphosphonic acid, salts thereof, and mixtures thereof.

8. A hair styling composition according to claim 7, wherein the ethylenically unsaturated carboxylic acids are chosen from acrylic acid, methacrylic acid, itaconic acid, fumaric acid, crotonic acid, and maleic acid.

9. A hair styling composition according to claim 6, wherein the hydrophilic non-ionic monomers are chosen from acrylamide, C₁₋₆ N-alkylated acrylamides, C₁₋₃ N,N-dialkylated acrylamides, polyethylene glycol acrylate, polyethylene glycol methacrylate, N-vinyl acetamide, N-methyl-N-vinyl acetamide, N-vinyl formamide, N-methyl-N-vinyl formamide, N-vinyl lactames comprising a cyclic group comprising from 4 to 9 carbon atoms, vinyl alcohol, ethylene oxide, hydroxyethyl acrylate, hydroxypropyl acrylate, hydroxyethyl methacrylate, hydroxypropyl methacrylate, methyl methacrylate, and N-acryloyl morpholine.

10. A hair styling composition according to claim 9, wherein the vinyl alcohol is copolymerized as a vinyl acetate and subsequently hydrolyzed.

11. A hair styling composition according to claim 6, wherein the hydrophilic cationic monomers are chosen from dimethyldiallyl ammonium chloride, methylvinyl imidazolium chloride, 2-vinylpyridine, 4-vinylpyridine, 2-methyl-5-vinylpyridine, N-(C₁₋₄ alkyl)-4-vinylpyridinium halides, vinyl amine, and monomers of formula:
H₂C═CR₁—CO—X₂
wherein:
R₁ is chosen from hydrogen and methyl,
X₂ is chosen from linear or branched C₁₋₆ hydrocarbyl groups bearing at least one entity chosen from primary amine functionalities, secondary amine functionalities, tertiary amine functionalities, quatemary nitrogen atoms, NHR₂ groups, and NR₂R₃ groups, wherein R₂ and R₃, which may be identical or different, are chosen from linear or branched C₁₋₆ hydrocarbyl groups bearing at least one entity chosen from primary amine functionalities, secondary amine functionalities, tertiary amine functionalities, and quaternary nitrogen atoms.

12. A hair styling composition according to claim 1, wherein the hydrophilic polymeric high porosity material comprises 0.1 to 40% by weight of at least one hydrophobic monomer chosen from vinyl aromatic monomers, dienes, diene alkylated derivatives, chloroprene, C₁-₁₀ alkyl acrylates, C₆₋₁₀ aryl acrylates, C₇₋₂₀ aralkyl acrylates, C₁₋₁₀ alkyl methacrylates, C₆₋₁₀ aryl methacrylates, C₇₋₂₀ aralkyl methacrylates, vinyl acetate, vinyl ethers of formula CH₂═CH—O—R, and ally ethers of formula CH₂═CH—CH₂—O—R, wherein R is chosen from C₁₋₆ alkyl groups, acrylonitrile, vinyl chloride, vinylidene chloride, caprolactone, ethylene, propylene, fluorinated vinyl monomers, perfluorated chain monomers, and mixtures thereof.

13. A hair styling composition according to claim 1, wherein the fixing polymer is chosen from anionic, cationic, amphoteric, and non-ionic fixing polymers.

14. A hair styling composition according to claim 1, further comprising a compound chosen from silicones, thickening agents, non-ionic surfactants, anionic surfactants, cationic surfactants, amphoteric surfactants, conditioning agents, softening agents, anti-foaming agents, moisturizing agents, emollients, plasticizers, water-soluble or oil-soluble, silicone or non-silicone sunscreens, permanent dyes, temporary dyes, organic pigments, inorganic pigments, inorganic fillers, clays, pearlescent agents, opacifying agents, colloids, fragrances, peptizing agents, preserving agents, ceramides, pseudo-ceramides, vitamins, provitamins, proteins, sequestering agents, solubilizing agents, alkalizing agents, anti-corrosion agents, fats such as vegetable, animal, mineral, and synthetic oils, reducing agents, antioxidants, oxidants, and mixtures thereof.

15. A hair styling composition according to claim 14, wherein the silicone is in a form chosen from soluble, dispersed, microdispersed, nanodispersed, volatile, and non-volatile forms.

16. A hair styling composition according to claim 1, wherein the physiologically acceptable liquid medium is chosen from water, at least one organic solvent, and mixtures of water and at least one organic solvent.

17. A hair styling composition according to claim 16, wherein the at least one organic solvent is chosen from C₁₋₄ alcohols, C₅₋₁₀ alkanes, fatty alcohols, optionally modified polyvalent alcohols, glycols, volatile or non-volatile silicones, mineral oils, organic oils, vegetable oils, and mixtures thereof.

18. A method for fixing a hair style, comprising applying a cosmetic composition to the hair, wherein the composition comprises, in a physiologically acceptable liquid medium:
at least one hydrophilic polymeric high porosity material, and
at least one fixing polymer,
with the proviso that the at least one hydrophilic polymeric high porosity material is not a silicone polymer or an alkyl polymethacrylate polymer (PMMA).
